(19) United States
(12) Patent Application Publication
(10) Pub. No.: US 2016/0346076 A1
(43) Pub. Date: Dec. 1, 2016

(54) CONTROLLED AXIAL DISPLACEMENT POSTERIOR CHAMBER PHAKIC INTRAOCULAR LENS

(71) Applicant: STAAR SURGICAL COMPANY, Monrovia, CA (US)

(72) Inventors: Thomas R. Paul, Westlake Village, CA (US); Alexi Ossipov, Laguna Niguel, CA (US); Jose L. Tlaxca, Gardena, CA (US)

(21) Appl. No.: 15/166,117

(22) Filed: May 26, 2016

Related U.S. Application Data
(60) Provisional application No. 62/166,226, filed on May 26, 2015.

(51) Int. Cl. A61F 2/16

(52) U.S. Cl. CPC A61F 2/161; A61F 2002/1689; A61F 2002/169

(57) ABSTRACT

An improved posterior chamber phakic intraocular lens (PCPIL) is provided. The improved PCPIL incorporates one or more design elements that minimize or eliminate axial displacement of the PCPIL under horizontal compression.

### CROSS-REFERENCE TO RELATED APPLICATIONS

This application claims priority to the earlier filed U.S. Provisional Application No. 62/166,226 filed on May 26, 2015, entitled “Controlled Vault ICL” which is incorporated reference herein in its entirety.

### BACKGROUND

The invention is generally directed to the field of treatment of visual deficiency, such as myopia, hyperopia, and astigmatism, either alone, or in combination with myopia or hyperopia. More specifically, the invention is directed to an improved haptic and/or footplate for an posterior chamber phakic intraocular lens (PCPIL).

As shown in FIG. 1, a PCPIL 5 intended to treat myopia or hyperopia, with or without astigmatism (also known as cylinder). PCPILs typically have a spherical power ranging from +15.0 Diopter (D) to −25.0 D with cylinder power with a magnitude up to about 10 D.

A current PCPIL typically has an optic zone or portion 7 surrounded by a haptic area 12. The PCPIL also has a spherical back radius 10 for both the haptics and optic designed to allow the PCPIL to be applied over the anterior surface of a patient's crystalline lens 30. (FIG. 2). Additionally, the PCPIL has a footplate 15 configured to be implanted in the sulcus 25 (FIG. 2) of the eye. In some variations, one or more tabs 20 may be disposed on the footplate. (FIG. 3). The planar footplates are typically arranged so that the footplates of an uncompressed PCPIL are in the horizontal plane. The spherical back radius of the PCPIL allows the lens, after implantation, to vault over the crystalline lens and avoid touching the crystalline lens 30 of the eye.

The spherical back radius 10 of the PCPIL also contributes to the optical power of the lens. Implantation of the PCPIL into the eye typically results in a compressive, horizontal force being applied to the footplates and haptics of the lens by the eye. Due to the design of the haptics and footplates, this compressive force has been found to cause the lens to displace axially in an anterior direction. This may be disadvantageous because such an axial displacement may cause, as an example, but not limited to, the anterior surface of the PCPIL pushing the iris of the eye anteriorly to the extent that draining of the aqueous through the angle of the eye could become restricted and the pressure in the anterior chamber of the eye could increase.

As seen in FIG. 4, the axial displacement of a prior art PCPIL as a function of horizontal compression is predictable. One method of controlling the axial displacement of the PCPIL during and after implantation has been to provide the PCPIL in a variety of sizes to accommodate various size eyes. However, this method requires the implanting surgeon to accurately estimate the diameter of the sulcus of the eye, a region of the eye that is not directly visible from outside of the eye and that varies from patient to patient, and then select the appropriate PCPIL size, which can be difficult. In view of this problem, it would be very desirable to have a PCPIL haptic and footplate design that minimizes lens axial displacement as a function of horizontal compression.

Moreover, if the PCPIL displaces axially in an uncontrolled manner when implanted, the positioning of the PCPIL within the eye may affect the precision of focus provided by the PCPIL as the effect of the lens is influenced by its proximity to other optical elements within the eye, including the cornea, the crystalline lens and the retina. This may result in a less than optimal visual outcome after implantation.

While an axial displacement that is too great may cause other problems within the eye as well, a PCPIL with too little clearance above the crystalline lens may also be problematic, as such a PCPIL may then contact the crystalline lens.

As is well known, the diameter of the eye available in which to implant a PCPIL can vary from eye to eye. Accordingly, an implanting physician attempts to control the amount of axial displacement of an implanted PCPIL by estimating the size of the eye, and then selects a PCPIL having an appropriate length. In many cases, however, the size of the eye and PCPIL cannot be identically matched, resulting in some residual compressive force on the haptics of the PCPIL, which causes the PCPIL to displace axially.

What has been needed, and heretofore unavailable, is a haptic and footplate design for use with a PCPIL that minimizes or eliminates PCPIL axial displacement as a function of horizontal compression. Further, such a design should improve the ability to properly size and implant the PCPIL such that any axial displacement of the PCPIL after implantation is controlled so as to prevent contact of the PCPIL with either the iris or crystalline lens of the eye. Such an improved PCPIL will also provide for easier and more accurate selection of the appropriate optical power of the PCPIL prior implantation so as to provide more predictable post-operative visual acuity. The present invention satisfies these, and other needs.

### SUMMARY OF THE INVENTION

In a general aspect, the present invention includes an improved design of the haptics and/or footplates of a PCPIL to minimize or eliminate axial displacement of the PCPIL when the PCPIL is placed under horizontal compression, such as occurs when the PCPIL is implanted in an eye. The improved PCPIL allows the initial axial displacement of the PCPIL to be independent of the overall length of the PCPIL, resulting in the axial displacement of the lens being minimized as the lens is horizontally compressed during implantation. Additionally, the improved PCPIL haptic and footplate design potentially reduces the number of PCPIL lengths that must be kept in inventory to treat a reasonable range of patients. Furthermore, the improvements allow the development of low axial displacement and high axial displacement PCPILs to meet individual patient needs.

In another aspect, the present invention includes an improved posterior chamber phakic intraocular lens, comprising: an optic; at least two supporting elements, each supporting element mounted to the optic on a diametrically opposed side of the optic; and a footplate disposed at a distal end of each supporting element, the footplate having an angulation that causes the footplate to bend anteriorly when the footplate and support elements are placed under horizontal compression.

In still another aspect, the present invention includes an improved posterior chamber phakic intraocular lens, comprising: an optic; and at least two supporting elements, each supporting elements having a length and a proximal end mounted to the optic on a diametrically opposed side of the optic, each of the supporting elements also having a footplate disposed at a distal end of the haptic, and each of the supporting elements also having a bending zone disposed along the length of the supporting element and disposed between the proximal and distal ends of the supporting element. In an alternative aspect, the bending zone includes a hinge-like portion. In another alternative aspect, the bending zone includes a compression element. In still another alternative aspect, the bending zone includes a section of the length of the supporting element having a thinner cross-section than the cross-section of the remainder of the length of the supporting element.

In yet another aspect, the present invention includes an improved posterior chamber phakic intraocular lens, comprising: an optic; a haptic body surrounding the optic, the haptic body having a first side and a second side, the first and second sides located on opposite sides of the optic along a longitudinal axis; a slit or opening disposed within each of the first and second sides of the haptic body; and at least two supporting elements, each supporting elements having a length and a proximal end mounted to the haptic body on a diametrically opposed side of the optic, each of the supporting elements having a distal end having an anterior angulation ranging from greater than 0 degree to 45 degree relative to a planar surface.

In still another aspect, the present invention includes an improved posterior chamber phakic intraocular lens, comprising: an optic; a haptic body surrounding the optic, the haptic body having a first side and a second side, the first and second sides located on opposite sides of the optic along a longitudinal axis; and at least two supporting elements, each supporting element having a length and a proximal end mounted to the haptic on a diametrically opposed side of the optic, each of the supporting elements configured to deform when compressed so that axial displacement of the optic is minimized due to the compression of lens. In one alternative aspect, the supporting element has an anterior angulation ranging from greater than 0 degrees to 45 degrees. In another alternative aspect, the supporting element tapers from a first thickness at a proximal end to a distal end having a second thickness less than the first thickness. In yet another alternative aspect, the support element tapers from a first thickness at a distal end to a proximal end having a second thickness less than the first thickness. In still another alternative aspect, the supporting element has a distal portion that curves anteriorly. In yet another alternative aspect, the supporting element includes a plurality of grooves disposed on an anterior surface of the supporting element. In still another alternative, the lens includes a slit or opening disposed within each of the first and second sides of the haptic body.

In another aspect, the present invention includes an improved posterior chamber phakic intraocular lens, comprising: an optic; a haptic body surrounding the optic, the haptic body having a posterior and an anterior side, the posterior side having a non-spherical curvature similar to the curvature of the crystalline lens of an eye; and a first side and a second side, the first and second sides located on opposite sides of the optic along a longitudinal axis; and at least two supporting elements, each supporting elements having a length and a proximal end mounted to the haptic body on a diametrically opposed side of the optic, each of the supporting elements also having at least one tab disposed at a distal end of the supporting element.

In still another aspect, the present invention includes an improved posterior chamber phakic intraocular lens, comprising: an optic; a haptic body surrounding the optic; at least two supporting elements, each supporting elements having a length and a proximal end mounted to the haptic body on a diametrically opposed side of the optic; and a notch disposed on an anterior side of a junction formed between at least one of the supporting elements and the haptic body.

In yet another aspect, the present invention includes an improved posterior chamber phakic intraocular lens, comprising: an optic; a haptic area; at least two supporting elements, each supporting element mounted to the haptic area on a diametrically opposed side of the haptic area; and a pair of footplates, each footplate having a proximal end joined to one of the two supporting elements, the each footplate having an anterior angulation relative to a planar surface such that the footplate deforms anteriorly when the footplates are placed under horizontal compression. In one alternative aspect, the anterior angulation is selected from the range of greater than 0 degrees and less than 90 degrees. In yet another alternative aspect, the anterior angulation is selected from the range of greater than 0 degrees and less than 45 degrees. In another alternative aspect, the anterior angulation is between 3 and 15 degrees. In still another alternative aspect, the anterior angulation is between 4 and 6 degrees.

In another aspect, the present invention includes an improved posterior chamber phakic intraocular lens, comprising: an optic; a haptic area; and at least two supporting elements, each supporting elements having a length and a proximal end mounted to the haptic area on a diametrically opposed side of the optic, each of the supporting elements also having a distal end, and each of the supporting elements also having a bending zone disposed along the length of the supporting element and disposed between the proximal and distal ends of the supporting element. In another aspect, the bending zone includes a hinge-like portion. In yet another aspect, the bending zone includes a compression element. In still another aspect, the bending zone includes a section of the length of at least one of the supporting elements having a thinner cross-section than the cross-section of the remainder of the length of the supporting element. In still another aspect, the bending zone is disposed along a length of the haptic area. In still another aspect, the at least two supporting elements are anteriorly angled with respect to the haptic area.

In another aspect, the present invention includes an improved posterior chamber phakic intraocular lens, comprising: an optic; a haptic body surrounding the optic, the haptic body having a first side and a second side, the first and second sides located on opposite sides of the optic along a longitudinal axis; and at least two footplates, each footplate having a length and a proximal end mounted to the haptic body on a diametrically opposed side of the optic, each of the footplates having a portion configured to deform when compressed so that axial displacement of the optic is minimized due to the compression. In one aspect, at least one of the at least two footplates has an anterior angulation ranging from more than 0 degrees to less than 90 degrees. In another aspect, at least one of the at least two footplates has an anterior angulation of greater than 0 degrees and less than 45 degrees. In another alternative aspect, at least one of the at least two footplates has an anterior angulation of between 3 and 15 degrees. In yet another aspect, at least one of the at least two footplates has an anterior angulation of between 4 and 6 degrees. In still another aspect, at least one of the at least two footplates tapers from a first thickness at a proximal end to a distal end having a second thickness less than the first thickness. In still another aspect, at least one of the at least two footplates tapers from a first thickness at a distal end to a proximal end having a second thickness less than the first thickness. In yet another aspect, at least one of the at least two footplates has a distal portion that curves anteriorly. In still another aspect, at least one of the at least two footplates includes a plurality of grooves disposed on an anterior surface of the footplate. In a further aspect, the improved posterior chamber phakic intraocular lens of claim 11, further comprises a slit or opening disposed on an anterior surface of the haptic body. In even another aspect, the haptic body has a first thickness, and the proximal end of at least one of the at least two footplates has a second thickness such that a ratio of the first thickness to the second thickness is between is greater than 1.0 and less than 2.0. In yet another aspect, the haptic body has a first thickness, and the proximal end of at least one of the at least two footplates has a second thickness such that a ratio of the first thickness to the second thickness is greater than 1.25 and less than 1.75. In another aspect, the haptic body has a first thickness, and the proximal end of at least one of the at least two footplates has a second thickness such that a ratio of the first thickness to the second thickness is between is greater than 1.4 and less than 1.6.

In another aspect, the present invention includes an improved posterior chamber phakic intraocular lens, comprising: an optic; a haptic body surrounding the optic, the haptic body having a posterior and an anterior surface, the posterior surface having a non-spherical curvature similar to the curvature of the crystalline lens of an eye; and at least two supporting elements, each supporting elements having a length and a proximal end mounted to the haptic body on a diametrically opposed side of the optic, each of the supporting elements also having a footplate disposed at a distal end of the haptic body. In another aspect, at least one of the at least two supporting elements has a distal end that is angled anteriorly with respect to the haptic body. In yet another aspect, the distal end of the at least one of the at least two supporting elements has an angulation configured to absorb compressive force applied to the at least two supporting elements so as to reduce anterior axial displacement of the optic resulting from application of the compressive force to the at least two supporting elements.

In still another aspect, the present invention includes an improved posterior chamber phakic intraocular lens, comprising: an optic; a haptic body surrounding the optic; at least two supporting elements, each supporting elements having a length and a proximal end mounted to the optic on a diametrically opposed side of the optic, each of the supporting elements; and a notch disposed on an anterior side of a junction between the haptic body and at least one of the two supporting elements and the haptic.

Other features and advantages of the invention will become apparent from the following detailed description, taken in conjunction with the accompanying drawings, which illustrate, by way of example, the features of the invention.

### BRIEF DESCRIPTION OF THE DRAWINGS

FIG. 1 is a cross-sectional view of a PCPIL intended for implantation within an eye.

FIG. 2 is a cross-sectional view of the PCPIL of FIG. 1 implanted in an eye.

FIG. 3 is a top view of the PCPIL of FIG. 1 illustrating the optic portion, haptics, and footplates of the PCPIL.

FIG. 4 is a graph illustrating the function of axial displacement as a function of compression distance for a series of PCPILs.

FIG. 5 is a cross-section view of an embodiment of the present invention depicting a PCPIL having an upwardly angled footplate.

FIG. 6A is a graph illustrating a comparison of axial displacement as a function of compression of a prior art PCPIL and the PCPIL of FIG. 5.

FIG. 6B is an enlarged view of the graph of FIG. 6A illustrating axial displacement as a function of compression of the PCPIL of FIG. 5.

FIG. 7 is a cross-section view of an embodiment of the present invention depicting a PCPIL having a compression element disposed between a haptic area and a footplate.

FIG. 8 is a cross-section view of an embodiment of the present invention depicting a PCPIL having a hinge-like portion disposed on a posterior surface of a haptic area or footplate of a PCPIL.

FIG. 9 is a cross-section view of an embodiment of the present invention depicting a PCPIL having haptic portion that has a reduced thickness compared to other portions of the haptic area of the PCPIL

FIG. 10 is a top view of a PCPIL similar to the embodiments of FIGS. 7-9 and including slits or openings formed on an interior surface of the PCPIL.

FIG. 11 is a top view of a PCPIL having total or partial thickness openings formed in the anterior surface of the PCPIL.

FIG. 12A is a cross-section view of an embodiment of the present invention depicting a PCPIL having a notch formed in an anterior surface of the PCPIL disposed between a haptic area and a footplate.

FIGS. 12B and 12C are enlarged views of the ends of the embodiment of FIG. 12A.

FIG. 13 is a cross-section view of an embodiment of the present invention depicting a PCPIL having a haptic area having a portion that is thicker than the same haptic area as depicted in FIG. 12A.

FIG. 14 is a cross-section view of an embodiment of the present invention depicting a PCPIL having a footplate having a thickness less that the same footplate depicted in FIG. 13.

FIG. 15A is a cross-section view of an embodiment of the present invention depicting a PCPIL having a footplate and tab that tapers to a maximal thickness located at a distal end of the footplate.

FIGS. 15B and 15C are enlarged views of the ends of the embodiment of FIG. 15A.

FIG. 16A is a cross-section view of an embodiment of the present invention depicting a PCPIL having a footplate that tapers from a maximal thickness at a proximal end of the footplate to a minimal thickness at a distal end of the footplate.

FIGS. 16B and 16 are enlarged views of the ends of the embodiment of FIG. 16A.

FIG. 17A is a cross-section view of an embodiment of the present invention depicting a PCPIL having a footplate having a portion that curves anteriorly.

FIGS. 17B and 17C are enlarged views of the ends of the embodiment of FIG. 17A.

FIG. 18A is a cross-section view of an embodiment of the present invention depicting a PCPIL having a footplate that includes grooves formed on an anterior surface of the footplate.

FIGS. 18B and 18C are enlarged views of the ends of the embodiment of FIG. 18A.

FIG. 19 is a graphical representation illustrating the effect on axial displacement as a function of the asphericity of the posterior radius of curvature of a PCPIL.

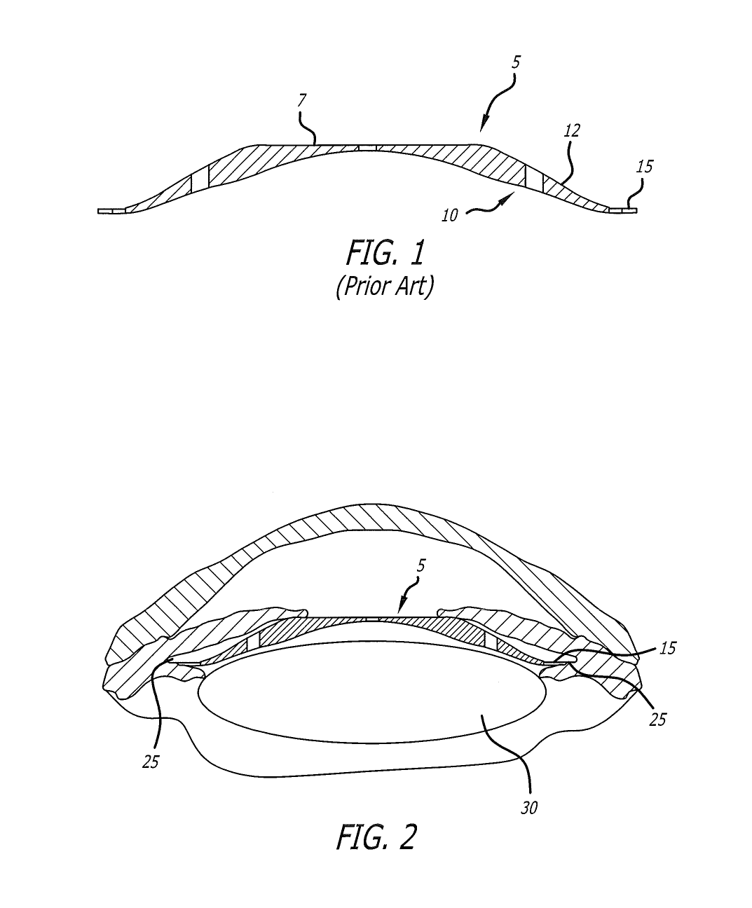

### DETAILED DESCRIPTION OF THE PREFERRED EMBODIMENTS

In the following description, numerous specific details are set forth in order to provide a thorough understanding of the present invention. It will be apparent, however, to one of ordinary skill in the art, that the present invention may be practiced without these specific details. In other instances, well known components or methods have not been described in detail but rather in a block diagram, or a schematic, in order to avoid unnecessarily obscuring the present invention. Further specific numeric references such as “first driver,” may be made. However, the specific numeric reference should not be interpreted as a literal sequential order but rather interpreted that the “first driver” is different than a “second driver.” Thus, the specific details set forth are merely exemplary. The specific details may be varied from and still be contemplated to be within the spirit and scope of the present invention.

This invention comprises multiple elements of a PCPIL haptic design that will individually and cumulatively minimize or eliminate PCPIL axial displacement as a function of horizontal lens compression.

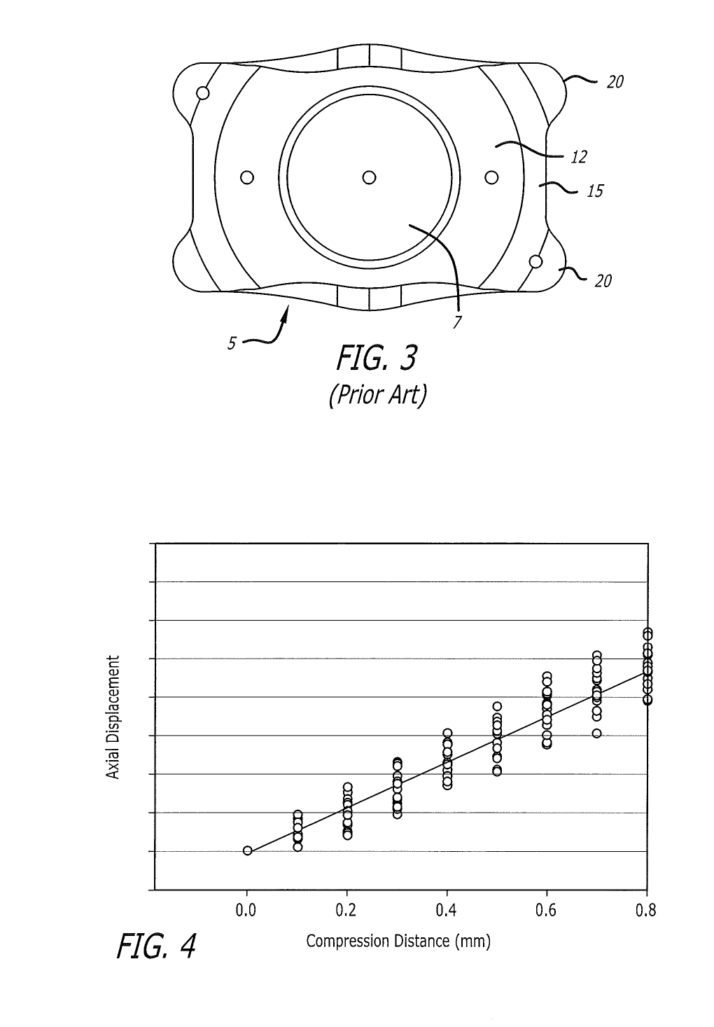

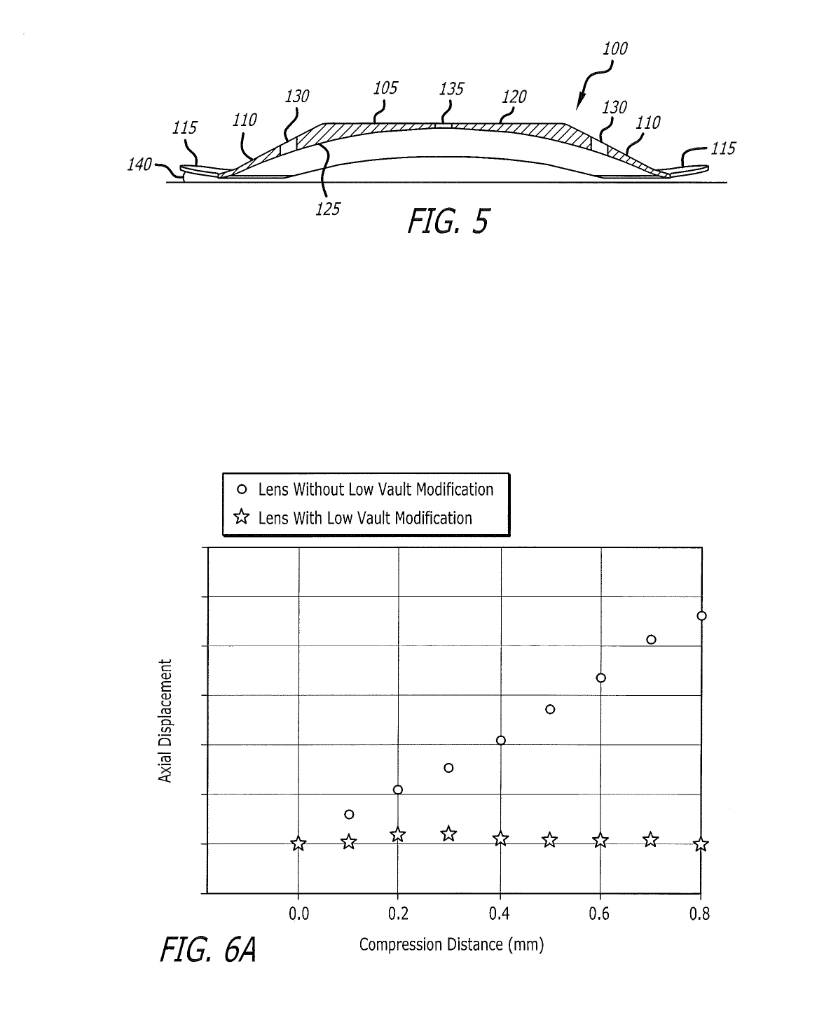

FIG. 5 is a cross-section view of one embodiment of a PCPIL 100 having an improved haptic and footplate design in accordance with the present invention. PCPIL 100 has an optic zone or portion 105 surrounded by a haptic area 110. Disposed around the haptic area is at least one supporting element, or footplate(s) 115. As shown, the footplates are located at opposite sides of the PCPIL. As shown in FIG. 3, the footplate may optionally include one or more tabs disposed at distal ends of the footplates. For example, there may be no tabs, one tab, two pads, three pads, four tabs, or more tabs depending on the design requirements of the PCPIL.

As can be seen in FIG. 5, the PCPIL has an anterior side 120 and a posterior side 125. The PCPIL may also have, but not necessarily, one or more holes 130 extending from the anterior side to the posterior side of the PCPIL disposed in the haptic area 110. The PCPIL may also have, but not necessarily, one or more holes 135 extending from the anterior side to the posterior side of the PCPIL disposed in the optic zone or portion 105. For example, the PCPIL may have a hole 135 located in a center of the optic zone or portion 105. These holes may, for example, provide for equalization of fluid volume and/or pressure between the anterior and posterior surfaces of the PCPIL.

Footplates 115 have a proximal end attached to the haptic area 110 and a distal end that is designed to be implanted into the eye. In this embodiment, the footplates are not disposed on a horizontal plane. Rather, the distal ends of the footplates are angled anteriorly at an angle 140 such that the distal end of the footplate is angled towards the anterior side of the PCPIL. The addition of the angulation 140 allows the distal end of the footplates to bend anteriorly when a compression force is imparted to the footplate. This upward angulation thus allows the PCPIL to be compressed when the PCPIL is implanted while eliminating or minimizing the amount of axial displacement of the PCPIL. Those skilled in the art will understand that the amount of angulation of the footplate may be varied depending on the overall design parameters of the PCPIL to ensure that axial displacement of the PCPIL in response to a compressive force on the footplate is minimized or eliminated without departing from the intended scope of the invention. For example, the inventors have observed that the angulation of the footplate relative to a planar surface can range from, for example, greater than 0 degrees to less than 90 degrees; or the angulation may range from greater than 0 degrees to less than or equal to 45 degrees; or the angulation may range between 3 and 15 degrees; or the angulation may range from 4 degrees to 6 degrees; or the angulation may be approximately 5 degrees.

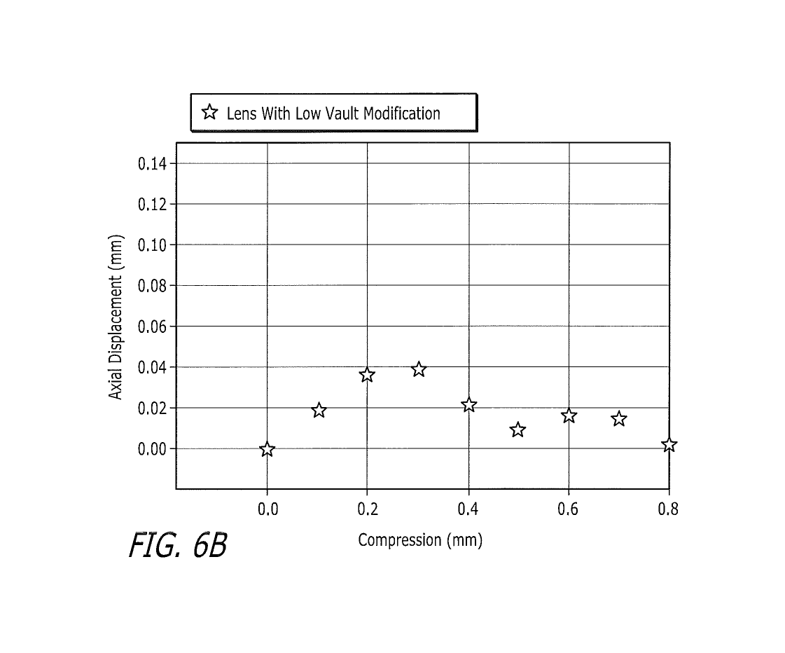

The result of the angulation added to the footplates as discussed above is illustrated in FIG. 6A, which depicts a comparison of the axial displacement as a function of distance of compression for a prior art PCPIL and an improved PCPIL in accordance with one embodiment of the invention having footplates that are angled five degrees anteriorly from the horizontal plane. FIG. 6B is an enlarged view of the view of FIG. 6A depicting the axial displacement performance of only the improved PCPIL.

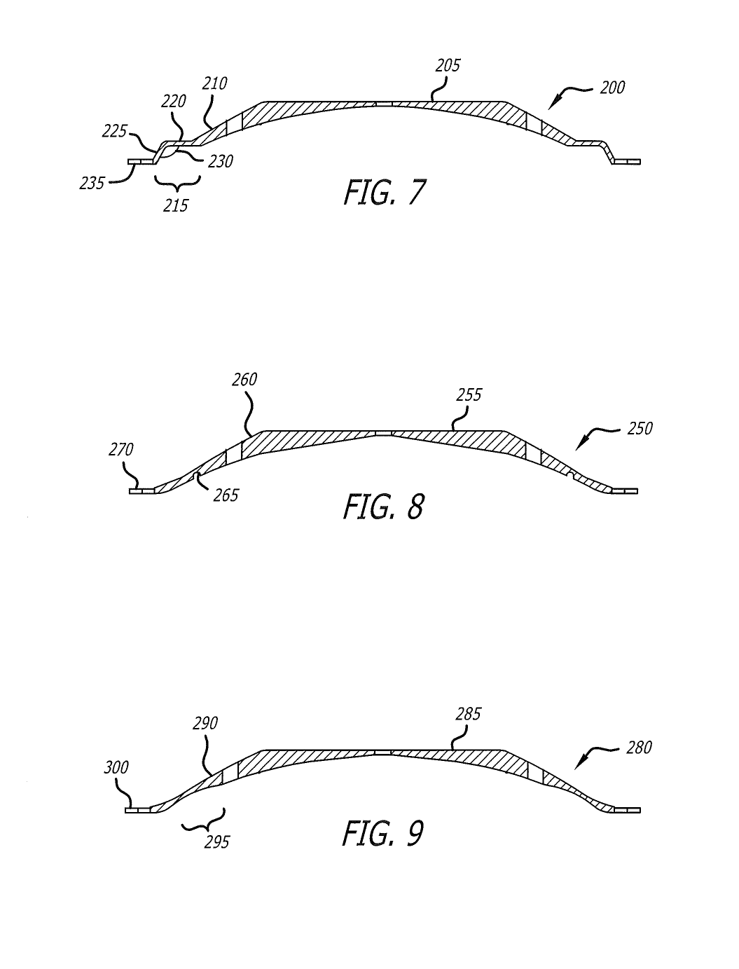

FIG. 7 is alternative embodiment in accordance with the present invention illustrating a PCPIL 200 having an optic zone or portion 205 and a haptic area 210. A compression element 215 is disposed between the haptic area 210 and footplate 235. In this embodiment, compressive element 215 has a proximal portion 220 attached to haptic area 210 and a distal portion 225 attached to footplate 235. Proximal portion 220 and distal portion 225 are joined in such a manner so that there is an angle 230 formed at the junction between them. Compression of the PCPIL at the distal end of the footplate 235 causes the compression element 215 to bend while imparting little or no axial displacement to the PCPIL. Those skilled in the art will understand that the amount of angle 230 may be varied depending on the overall design of the PCPIL without departing from the scope of the intended invention.

FIG. 8 is another alternative embodiment in accordance with the present invention illustrating a PCPIL 250 having an optic zone or portion 255 and a haptic area 260. In this embodiment, a hinge-like portion 265 is added to a posterior side of the haptic area 260. In one embodiment, the hinge-like portion is formed by decreasing the thickness of hinge-like portion such that when the haptic area experiences a compressive force, the haptic area deforms at the location of the hinge-like portion in such a way that little if any axial displacement is imparted to the optic or zone portion of the PCPIL. The size and depth of the hinge-like portion may be adjusted as needed to minimize the axial displacement of the PCPIL as a function of compressive distance when the PCPIL is implanted in the eye. Other embodiments of hinge-like portions are possible. For example, but not limited to, a divot may be sculpted from the posterior or anterior surfaces of the haptic body. In another embodiment, the hinge-like portion may be formed in the anterior or posterior surface of one or more of the footplates of the PCPIL.

FIG. 9 is still another alternative embodiment in accordance with the present invention illustrating a PCPIL 280 having an optic zone or portion 285 and a haptic area 290. In this embodiment, the haptic area has at least one portion 295 having a thickness thinner than other portions of the haptic area. The inclusion of portion 295 in the haptic area causes the haptic area to bend in the vicinity of portion 295 the when a compressive force is imparted to the tab 300 and haptic area. As illustrated, the thickness of portion 295 may not necessarily be the same along the length of portion 295, but may be contoured as desired to provide a desired amount of deformation when a compressive force is imparted to the footplate and haptic area to minimize the axial displacement of the PCPIL as a function of compressive distance when the PCPIL is implanted in the eye.

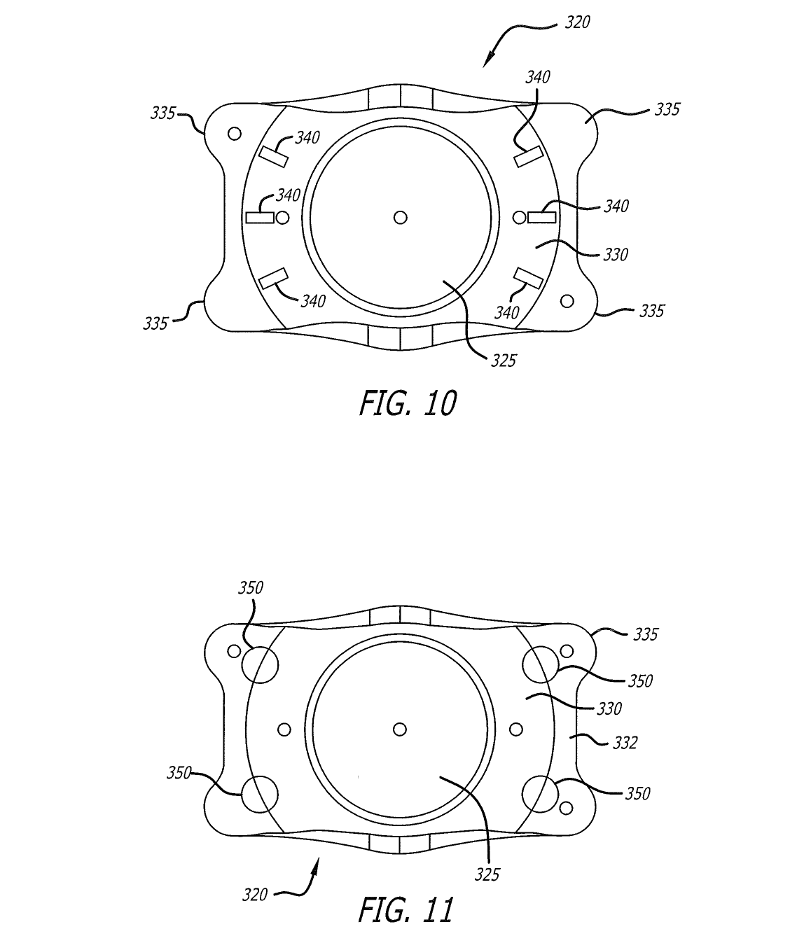

FIG. 10 illustrates an another embodiment of a PCPIL in accordance with the present invention. FIG. 10 depicts a PCPIL 320 having an optic zone or portion 325, a haptic area 330, and footplates 335. In this embodiment, one or more short vertical slits or openings 340 are disposed in the haptic area 330 on a radial axis relative to the optic zone or portion, and located at the approximate location of the compression element 215 described above (FIG. 7), hinge-like portion 265 (FIG. 8), or thin haptic portion 295 (FIG. 9). Slits or openings 340 allow the haptic area, whose posterior surface is a section of a sphere, to flex symmetrically without distortion or buckling when the haptic area is deformed by compression of the PCPIL.

FIG. 11 depicts the PCPIL 320 having an optic zone or portion 325, haptic area 330, and footplates 225. In this embodiment, holes 350 extending through the PCPIL are disposed across a junction between the haptic area 330 and footplate 332 adjacent to the footplates 335. Thus, a portion of the hole extends through the haptic area and another portion of the hole extends through the footplate. Such an arrangement allow the haptic and footplate to bend in a manner that results in reduced axial displacement of the PCPIL in response to compression when the PCPIL is implanted in the eye. In another embodiment, the hole does not need to extend through the haptic area and footplate; it may be a partial depth hole or depression disposed in either the anterior side of the PCPIL or the posterior side of the PCPIL. Alternatively, a depression may be formed on both sides of the PCPIL, but not extending through the PCPIL.

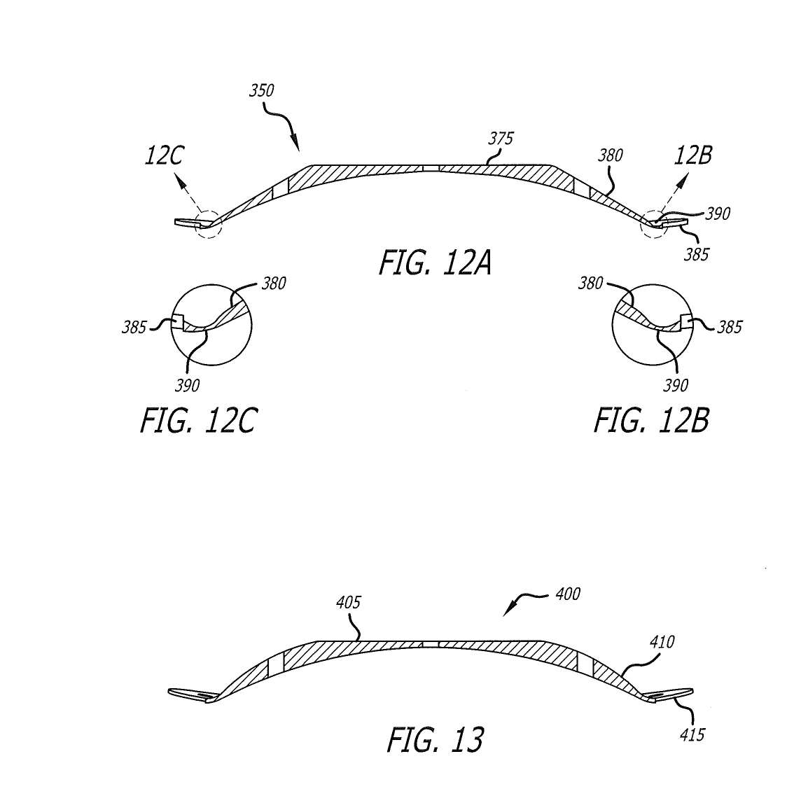

FIGS. 12A, 12B, and 12C illustrate another alternative embodiment in accordance with the present invention. In this embodiment, PCPIL 350 has an optic zone or portion 375, a haptic area 380 and footplate 385. A notch 390 is formed at the anterior side of the junction of the haptic area 380 and the footplate 385. The notch 390 encourages the distal end of footplate 385 to move anteriorly when the PCPIL is compressed upon implantation by reducing resistance to the bending of the footplate at the junction of the footplate and haptic area.

Note that while notches are shown at being formed at both sides of the PCPIL, the notches could be formed at only one side of the PCPIL. When “sides” is mentioned with respect to the PCPIL, reference is being made to the area of the PCPIL at which the footplates are located.

FIG. 13 illustrates an alternative embodiment in accordance with the present invention. In this embodiment, PCPIL 400 has an optic zone or portion 405, a haptic area 410 and an anteriorly angled footplate 415. Haptic area 410 is preferentially thickened along at least a portion of its length so as to resist bending of haptic area 410 when the PCPIL is compressed, thus assisting is urging the distal end of footplate 415 to move anteriorly to minimize axial displacement of the PCPIL when it is implanted.

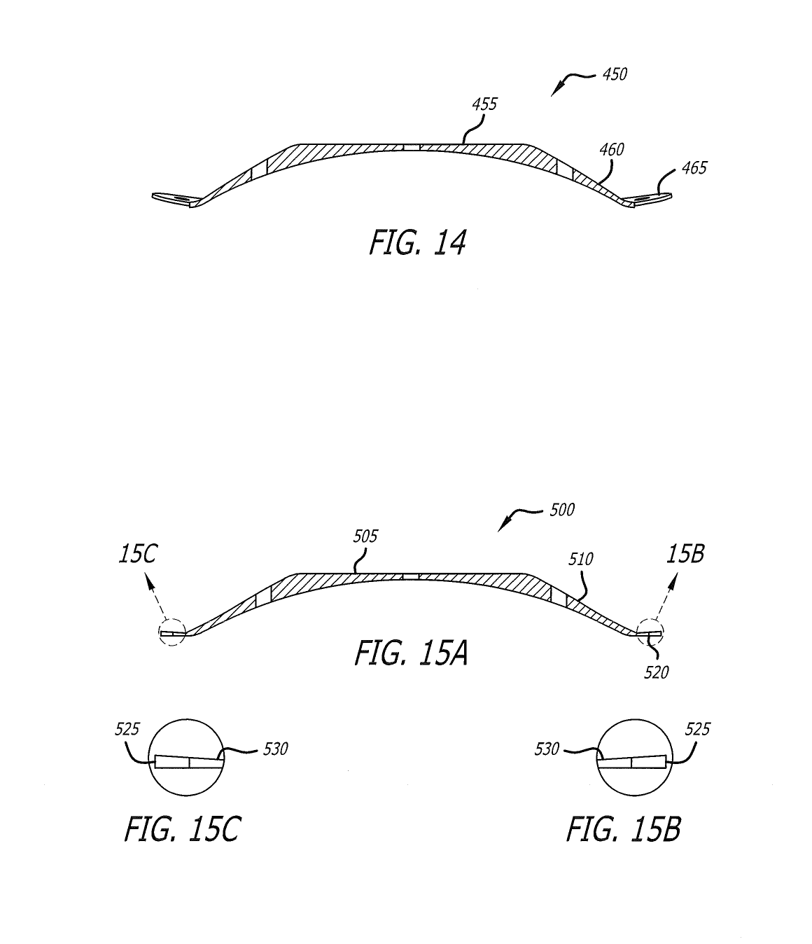

FIG. 14 illustrates an alternative embodiment in accordance with the present invention. In this embodiment, PCPIL 450 has an optic zone or portion 455, a haptic area 460 and an anteriorly angled footplate 465. In this embodiment the footplate is formed having a thickness less than the footplate 415 depicted in FIG. 13. The reduced thickness of footplate 465 is designed to encourage deformation of the footpad when PCPIL 450 is implanted such that axial displacement of the PCPIL is minimized.

FIGS. 15A, 15B, and 15C illustrate another alternative embodiment in accordance with the present invention. In this embodiment, PCPIL 500 has an optic zone or portion 505, a haptic area 510 and footplate 520. As shown more clearly in FIGS. 15B and 15C, the footplate has a proximal end 530 and a distal end 525. The footplate also has a thickness that tapers from a maximal thickness at the distal end 525 to the proximal end 530 where the footplate has a minimum thickness less than the thickness of the distal end 525. The tapered shape of the footplate 520 encourages distortion of the proximal end of the footplate and minimize axial displacement of the PCPIL when it is implanted in an eye.

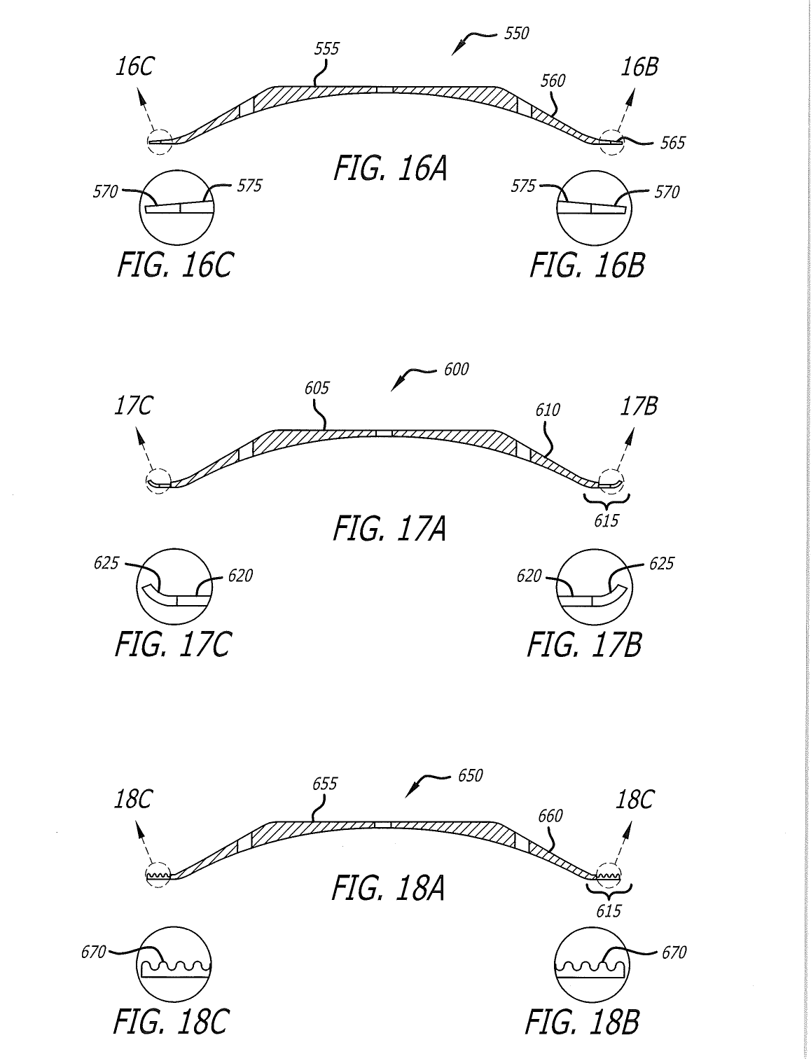

FIGS. 16A, 16B, and 16C illustrate another alternative embodiment in accordance with the present invention. In this embodiment, PCPIL 550 has an optic zone or portion 555, a haptic area 560 and footplate 565. As shown more clearly in FIGS. 16B and 16C, the footplate has a proximal end 575 and a distal end 570. The footplate also has a thickness that tapers from a maximal thickness at the proximal end 575 to the distal end 570 where the footplate has a minimum thickness less than the thickness of the proximal end 575. The tapered shape of the footplate aides in minimizing axial displacement of the PCPIL when it is implanted in an eye.

While several embodiments have been described where the thickness of the haptic area, or one or more portions of the haptic or footplates have been adjusted to control the axial displacement of the PCPIL in the presence of a compression force, those skilled will understand that other arrangements are possible to achieve the same result. The inventors have observed, for example, that reduction in the axial displacement of a PCIPL may be achieved where the ratio of haptic thickness to footplate thickness at the junction of the two is approximately 2.0 to 1.0, and preferably approximately 1.5. For example, for the embodiment of the improved PCPIL illustrated in FIGS. 6A and 6B, the nominal thickness of the haptic area was 104 microns, and the thickness of the footplate was 70 microns, giving a ratio of 1.49.

FIGS. 17A, 17B, and 17C illustrate another alternative embodiment in accordance with the present invention. In this embodiment, PCPIL 600 has an optic zone or portion 605, a haptic area 610 and footplate 615. As shown more clearly in FIGS. 17B and 17C, the footplate has a proximal portion 620 and a distal portion 625. The proximal portion is substantially straight while the distal portion of the footplate is curved anteriorly. When the distal portion is compressed when the PCPIL is implanted, the distal portion of the footplate is distorted in response to the force in a manner so as to minimize axial displacement of the PCPIL.

FIGS. 18A, 18B, and 18C illustrate another alternative embodiment in accordance with the present invention. In this embodiment, PCPIL 650 has an optic zone or portion 555, a haptic area 660 and footplate 665. As shown more clearly in FIGS. 18B and 18C, the footplate has a portion 665 on which grooves 670 are formed. While grooves 670 are typically formed on the anterior surface of portion 665, the grooves may also be formed on the posterior surface of portion 665. While the tetra “groove” is used, that term is meant to encompass any groove-like shape, such as serrations, channels, and the like. Any form applied to the footplate that results in preferential deformation of the footplate that reduces axial displacement of the PCIPL is intended to be within the scope of the present invention.

In still another embodiment, the posterior radius of curvature of the PCPIL haptic is modified to more closely match the anterior curvature of the human crystalline lens. The anterior surface of the human crystalline lens has more of a flat or elliptical curvature rather than a spherical curvature. Present PCPILs, on the other hand, have a spherical posterior radius. By making at least part of the central part of the PCPIL's posterior curvature to have a flattened or elliptical shape, the PCPIL will have less initial axial displacement. Additionally, this flatter posterior PCPIL design allows for the design of low initial axial displacement or high initial axial displacement PCPILs to accommodate different eye structures.

A flatter posterior PCPIL design contributes to a lower axial displacement of the lens as it is horizontally compressed during implantation. The previously described design elements can, of course, be applied to the flatter posterior curvature PCPIL to optimize the haptic performance and minimize or eliminate axial displacement.

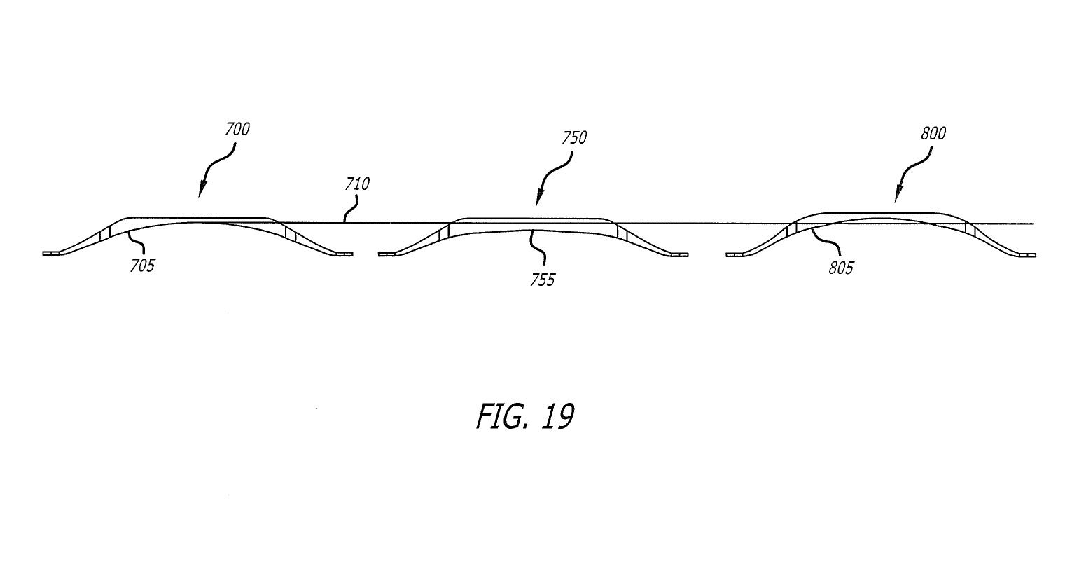

FIG. 19 illustrates the effect of altering the radius of curvature of the posterior surface of a PCPIL. PCPIL 700 has a posterior spherical radius of curvature 705, which is typical of prior art PCPILs. In contrast, improved PCPILs 750, 800 have a non-spherical posterior radius of curvature 755, 805 respectively. The effect on the axial displacement of each PCPIL as a function of the different radii of curvature is readily apparent when the PCPILs are compared to reference line 710.

PCPIL 750, which has a flatter aspheric posterior radius of curvature 755 has less initial axial displacement than PCPIL 700, which has a spherical posterior radius of curvature 705. Similarly, PCPIL 800, which has a steeper aspheric posterior radius of curvature 805, has a higher initial axial displacement than PCPIL 700.

Non-spherical or aspheric posterior surfaces of a PCPIL may be generated using a geometrical conic equation and varying the conic constant to achieve posterior shapes that assist in achieving predictable desirable axial displacement of a PCPIL. The equation for a conic section with an apex at the origin and tangent to the Y axis is:

Y²−2RX+(K+1)X²=0 Equation 1:

where K is the conic constant and R is the radius of curvature at X=0.

This formula is used to specify oblate elliptical (K>0) surfaces, spherical (K=0) surfaces, prolate elliptical (0>K>−1) surfaces, parabolic (K=−1) surfaces, and hyperbolic (K<−1) surfaces. By adjusting the conic constant and aspheric coefficients, an aspheric posterior surface can be optimized to adjust the amount of distance between the anterior surface of the crystalline lens and the posterior surface of a PCPIL.

While various embodiments of the present invention have been described individually, it should be understood that one or more, or all, of the embodiments may be combined to provide a PCPIL design that results in the elimination or minimization of the undesirable axial displacement when the PCPIL is compressed during implantation. The improved PCPIL described above allows the initial axial displacement of the PCPIL to be independent of the overall length of the PCPIL. Moreover, the various embodiments set forth above provide the resulting axial displacement of the lens to be minimized as the lens is horizontally compressed during implantation, and may also reduce the number of lengths of the PCPIL needed to treat a wide range of patients. Further, some embodiments allow the design and manufacture of low axial displacement and high axial displacement PCPILs to meet individual patient needs.

While several particular forms of the invention have been illustrated and described, it will be apparent that various modifications can be made without departing from the spirit and scope of the invention.

We claim:

1. An improved posterior chamber phakic intraocular lens, comprising:
an optic;
a haptic area;
at least two supporting elements, each supporting element mounted to the haptic area on a diametrically opposed side of the haptic area; each supporting element end having a proximal end joined to the haptic area, and each supporting element having an anterior angulation relative a planar surface such that the support element deforms anteriorly when the supporting elements are placed under horizontal compression.

2. The improved posterior chamber phakic intraocular lens of claim 1, wherein the anterior angulation is selected from the range of greater than 0 degrees and less than 90 degrees.

3. The improved posterior chamber phakic intraocular lens of claim 1, wherein the anterior angulation is selected from the range of greater than 0 degrees and less than 45 degrees.

4. The improved posterior chamber phakic intraocular lens of claim 1, wherein the anterior angulation is between 4 and 6 degrees.

6. The improved posterior chamber phakic intraocular lens of claim 1, wherein compressing the at least two supporting elements 0.6 mm results in a change of anterior axial displacement of the optic of less than 100 microns.

7. The improved posterior chamber phakic intraocular lens of claim 1, further comprising a hinge-like portion disposed on a posterior surface of the haptic area.

8. The improved posterior chamber phakic intraocular lens of claim 1, further comprising a compression element disposed along the length of at least one of the at least two supporting elements.

9. The improved posterior chamber phakic intraocular lens of claim 1, further comprising a notch disposed on an anterior side of a junction between the haptic area and at least one of the at least two supporting elements and the haptic body.

10. An improved implantable contact lens, comprising:
an optic;
a haptic area; and
at least two supporting elements, each supporting elements having a length and a proximal end mounted to the haptic area on a diametrically opposed side of the optic, each of the supporting elements also having a distal end, and each of the supporting elements also having a bending zone disposed along the length of the supporting element and disposed between the proximal and distal ends of the supporting element.

11. The improved posterior chamber phakic intraocular lens of claim 10, wherein the bending zone includes a hinge-like portion.

12. The improved posterior chamber phakic intraocular lens of claim 10, wherein the bending zone includes a compression element.

13. The improved posterior chamber phakic intraocular lens of claim 10, wherein the bending zone includes a section of the length of at least one of the supporting elements having a thinner cross-section than the cross-section of the remainder of the length of the supporting element.

14. The improved posterior chamber phakic intraocular lens of claim 10, wherein the bending zone is disposed along a length of the haptic area.

15. The improved posterior chamber phakic intraocular lens of claim 14, wherein the at least two supporting elements are anteriorly angled with respect to the haptic area.

16. An improved posterior chamber phakic intraocular lens, comprising:
an optic;
a haptic body surrounding the optic, the haptic body having a first side and a second side, the first and second sides located on opposite sides of the optic along a longitudinal axis; and
at least two footplates, each footplate having a length and a proximal end mounted to the haptic body on a diametrically opposed side of the optic, each of the footplates having a portion configured to deform when compressed so that axial displacement of the optic is minimized due to the compression.

17. The improved posterior chamber phakic intraocular lens of claim 16, wherein at least one of the at least two footplates has an anterior angulation ranging from more than 0 degrees to less than 90 degrees.

18. The improved posterior chamber phakic intraocular lens of claim 16, wherein at least one of the at least two footplates has an anterior angulation of greater than 0 degrees and less than 45 degrees.

19. The improved posterior chamber phakic intraocular lens of claim 16, wherein at least one of the at least two footplates has an anterior angulation of between 3 and 15 degrees.

20. The improved posterior chamber phakic intraocular lens of claim 16, wherein at least one of the at least two footplates has an anterior angulation of between 4 and 6 degrees.

21. The improved posterior chamber phakic intraocular lens of claim 16, wherein at least one of the at least two footplates tapers from a first thickness at a proximal end to a distal end having a second thickness less than the first thickness.

22. The improved posterior chamber phakic intraocular lens of claim 16, wherein at least one of the at least two footplates tapers from a first thickness at a distal end to a proximal end having a second thickness less than the first thickness.

23. The improved posterior chamber phakic intraocular lens of claim 16, wherein at least one of the at least two footplates has a distal portion that curves anteriorly.

24. The improved posterior chamber phakic intraocular lens of claim 16, wherein at least one of the at least two footplates includes a plurality of grooves disposed on an anterior surface of the footplate.

25. The improved posterior chamber phakic intraocular lens of claim 16, further comprising:
a slit or opening disposed on an anterior surface of the haptic body.

26. The improved posterior chamber phakic intraocular lens of claim 16, wherein the haptic body has a first thickness, and the proximal end of at least one of the at least two footplates has a second thickness such that a ratio of the first thickness to the second thickness is between is greater than 1.0 and less than 2.0.

27. The improved posterior chamber phakic intraocular lens of claim 16, wherein the haptic body has a first thickness, and the proximal end of at least one of the at least two footplates has a second thickness such that a ratio of the first thickness to the second thickness is greater than 1.25 and less than 1.75.

28. The improved posterior chamber phakic intraocular lens of claim 16, wherein the haptic body has a first thickness, and the proximal end of at least one of the at least two footplates has a second thickness such that a ratio of the first thickness to the second thickness is between is greater than 1.4 and less than 1.6.

29. The improved posterior chamber phakic intraocular lens of claim 16, further comprising a notch disposed on an anterior side of a junction between the haptic body and at least one of the at least two footplates and the haptic body.

30. An improved posterior chamber phakic intraocular lens, comprising:
an optic;
a haptic body surrounding the optic, the haptic body having a posterior and an anterior surface, the posterior surface having a non-spherical curvature similar to the curvature of the crystalline lens of an eye; and
at least two supporting elements, each supporting elements having a length and a proximal end mounted to the haptic body on a diametrically opposed side of the optic, each of the supporting elements also having a footplate disposed at a distal end of the haptic body.

31. The improved posterior chamber phakic intraocular lens of claim 30, wherein at least one of the at least two supporting elements has a distal end that is angled anteriorly with respect to the haptic body.

32. The improved posterior chamber phakic intraocular lens of claim 31, wherein the distal end of the at least one of the at least two supporting elements has an angulation configured to absorb compressive force applied to the at least two supporting elements so as to reduce anterior axial displacement of the optic resulting from application of the compressive force to the at least two supporting elements.

33. An improved posterior chamber phakic intraocular lens, comprising:
an optic;
a haptic body surrounding the optic;
at least two supporting elements, each supporting elements having a length and a proximal end mounted to the optic on a diametrically opposed side of the optic, each of the supporting elements; and
a notch disposed on an anterior side of a junction between the haptic body and at least one of the at least two supporting elements and the haptic body.
